(19) United States
(12) Patent Application Publication
(10) Pub. No.: US 2018/0325183 A1
(43) Pub. Date: Nov. 15, 2018

(54) Smart Electronic Vaporizer

(71) Applicants: Liji Huang, Santa Clara, CA (US); Chih-Chang Chen, Cupertino, CA (US)

(72) Inventors: Liji Huang, Santa Clara, CA (US); Chih-Chang Chen, Cupertino, CA (US)

(73) Assignee: Wisenstech Ltd., Santa Clara, CA (US)

(21) Appl. No.: 16/046,797

(22) Filed: Jul. 26, 2018

Related U.S. Application Data
(63) Continuation-in-part of application No. 14/639,824, filed on Mar. 5, 2015.

(51) Int. Cl. A24F 47/00; G01F 1/684; G01N 33/00

(52) U.S. Cl. CPC A24F 47/008; G01N 33/0036; G01F 1/6845

(57) ABSTRACT

The design and structure as well as the control scheme of a smart electronic vaporizer device having a micro-machined (a.k.a. MEMS, Micro Electro Mechanical Systems) mass flow sensor and control electronics that provide the vaporizing process in proportional to the user inhalation flowrate or strength for the best simulation of the experience for traditional cigarette. The device further incorporates a MEMS gas composition sensor that is coupled with the mass flow sensor to measure the user's respiratory health data, including but not limited to asthma status and metabolism related respiratory exchange rate. The device is further capable to relay the data to the designated mobile device and further to the designated cloud for big data process and sharing.

### BACKGROUND OF THE INVENTION

### 1. Field of the Invention

This invention relates to an electronic vaporizer and/or an electronic device that simulates the experience of a traditional cigarette. In particular, the invention relates to a smart electronics vaporizer that shall be equipped with electronic sensing and measurement elements as well as the electronic vaporizer which can transmit data to a mobile device and the data shall be further shared in designated clouds. This invention is specifically for a smart connected personalized electronic vaporizer equipped with the personal respiratory measurement elements. This invention is further related to micromachined silicon sensors or Micro Electro Mechanical Systems (MEMS) mass flow and gas sensing technology that measures the quality and quantity of gases. The present invention additionally relates to interim of things and the relay to the data clouding and analysis for which the smart electronic vaporizer can transmit the data to the designated cloud via the mobile devices.

### 2. Description of the Related Art

The global ban of smoking has greatly promotes the market of electronic cigarettes that have grown in approximately twenty billion US dollars according to the various market reports. The harmful chemicals in the cigarettes are considered to be the major facilitator for human respiratory disease; therefore a replacement for cigarettes shall be beneficial for human being. The first disclosure of a heated vaporizer could be traced back some eighty years ago by Joseph Robinson (U.S. Pat. No. 1,775,947), when he taught a vaporizing device for holding medicinal compounds which were electrically heated to produce vapors for inhalation for individual use. This device although it was not intended for a replacement of cigarette, the design had very much the same basic components as for today's electronic cigarette. In 1960s, Herbert A. Gilbert (U.S. Pat. No. 3,200,819) presented a new device for liquid vaporization and first used the term of smokeless non-tobacco cigarette. According to Gilbert, the device was intentionally for providing an article for the replacement of the cigarette and it was to provide a safe and harmless means for smoking by replacing burning tobacco with heated, moist, flavored air. Disclosures for improved electronic cigarettes were filed in the following years such as by Egilmex (U.S. Pat. No. 4,945,929), Gori (U.S. Pat. No. 4,945,931) and Losee at al. (U.S. Pat. No. 5,095,921), but the real products were not becoming popular until in year 2000s. The products disclosed by Lik Hon (U.S. Pat. No. 7,832,410; U.S. Pat. No. 8,375,957) are considered to be the market pioneer for the industry. Hon taught an invention relates to an electronic atomization cigarette which contains nicotine without harmful tar and thus significantly reduces the carcinogenic risk. In addition, the customers shall feel as if they were smoking and experiencing the same.

The current electronic cigarettes in the art are constituent with four major components: a liquid container that can be filled by liquid with or without nicotine, a vaporizer that utilizes an electrical heater to vaporize the liquid in the said container, an electronics that powers the heater and an inhaler that can consume the liquid vapors generated from the vaporizer. To simulate the performance of a traditional cigarette, a number of improvements have been proposed, a LED type of sensor or hat source was added to the non-inhalation end of the electronic cigarette such that light and ash can be imaged during the operation (M. Scatterday, U.S. Pat. No. 8,539,959); a thermal regulator was used for control of the heating fuel temperature and the heater was separated from the vapor chamber to reduce any potential contaminants released from the exhaust gas from the heater (Monsees et al. U.S. Pat. No. 8,925,555), aerosol generator for quick delivers of the vaporized smoke as disclosed for the better proximate to traditional cigarette (Nichols et al., U.S. Pat. No. 6,854,461). Sophisticated electronic control circuitry that can provide multiple power cycles such that the electrical smoking system shall be more closely simulated for replacement of traditional cigarettes (Fleischhauer et al. U.S. Pat. No. 6,040,560). The ON/OFF of the electronic cigarettes are normally controlled via a switch button, but improvement for simulating a traditional cigarette also utilizes a sensor to automatically switch on and off the electronics cigarette. When the user starts to inhale the simulated smoke, the air flow due to inhalation shall trigger the switch of the said sensor. A typical simple and reliable sensor can be a magnetic Reed switch (Lik Hon, U.S. Pat. No. 8,393,331) or an air flow sensor (G. Pan, U.S. Pat. No. 8205,622). A pressure sensor (L. Liao European Patent EP20100816778) is also proposed for use of detection the gas flow threshold that will be further trigger the power switch such that it will avoid the power malfunction due to vibration or external noise. A smoke sensor with multicolored LED light (Q. Liu, US Patent Pub. No. 2014/0053856) are also proposed as an improvement for the exhibiting the state of the power and battery information. Further improvement disclosed an embedded Bluetooth device in the electronic cigarette for connectivity with the mobile device to record the simulated smoking frequency (J. Qi, US Patent Pub. No. 2014/0202477).

Despite of all these efforts to provide better smoking experiences as compared to those that are well known by traditional cigarette consumers, the differences are still clearly registered by the current offers. According to the various surveys, electronics cigarette adoption rate was merely about 26% compared to the trial rate of about 45%. The low adoption rate was not only because of the tightening regulations and the brand recognition, but the consumer dissatisfaction was considered the top reason for the slow-down of the electronic cigarette growth.

Therefore it is desired to have a new article that shall provide the consumers with the similar experiences as those with the traditional cigarettes while it shall add additional incentives or benefits to the consumers. Among all the new features that are missing for the current products, the critical one is the constant vaporizing heating power that yielded a restricted flow of the vapor available with any inhalation performance which is substantially different from the process of a traditional cigarette where a stronger inhalation shall lead to a higher amount of consumption of the substance of the cigarette and vice versa. To this objective, the present invention shall disclose a new mass flow sensing measurement scheme as well as an electronic circuitry with feedback loops. The mass flow sensing capability shall yield precise measurement to the inhalation process and send the information to the electronics to adjust the power of the vaporization such that sufficient supply of the smoke can be guaranteed via the vaporization. The said mass flow sensor shall further be fused with a gas composition sensor which will register the respiratory pattern and provide respiratory related health data such as metabolism and respiratory related diseases such as asthma tracer to the customers. The smart electronics shall further be able to register the usage of the device and transmit the data to a mobile device or stationed one that shall record the device usage as well as vapor consumption, and further relay the data to the cloud for big data process.

### SUMMARY OF THE INVENTION

It is the objective of the present disclosure to provide the design and structure of a device for electronics vaporizer that can be primarily applied for better simulation of an electronic cigarette. The device is incorporated with a sensor fusion having both air mass flow metering and carbon dioxide gas concentration measurement, and a control electronics that shall utilize the measured air flowrate to proportionally control the heating power for the liquid vaporizer such that the inhalation flow/rate shall lead to a proportional liquid vaporization volume. The device shall also provide the consumer's respiratory information such as the inhalation flowrate and the exhalation carbon dioxide to oxygen concentration ratio for the user for references. The device shall have the capability of interfacing with a mobile device for recording and management as well as displaying the said information which can be further relayed to the destined cloud fir big data process and feedback to the corresponding data providers. Further this invention disclosed the detailed assembly of the said device.

In one preferred embodiment, the disclosed device is for an electronics vaporizer that can be applied to formulate into the configuration for simulating the experience of a traditional cigarette. In particular, the said device shall be capable to generate the vapors from the vaporizer that shall be proportional to the inhalation flowrate of the user. The said device shall have the capability of precisely metering the inhalation flowrate by a mass flow sensor and converted the precise flowrate into an electronic signal for the adjustment of the power of the heater that is dedicated to the vaporization of the desired liquid such that the desired amount of the vapor can be generated. The said device shall further have the capability of measurement of the gas concentration from the respiratory of the user and provide such information to the user via the local display or a wireless relay to a mobile device that shall be able to process the information and transform the information into the plain language for the user. The said mobile device shall further relay the information to the designated cloud for big data process which shall be useful for public health promotion as well as for the user's own benefits.

In another preferred embodiment, the said device with the capability of metering the flowrate of inhalation as well as exhalation is accomplished by a silicon mass flow sensor made via the micro electro mechanical system fabrication process having a fast response time in mini-seconds and wide dynamic range. The said mass flow sensor shall utilize the thermal dissipation sensing principle that shall be independent of the variations of the environmental parameters such as pressure and temperature. The said mass flow sensor can be the one disclosed by the same inventors (U.S. Pat. Nos. 7,752,910; 7,765,679). However the said mass flow sensor is preferred to be further integrated with a thermal conductivity sensing element and a thermal capacity sensing element for measurement of the carbon dioxide gas composition from the inhalation or the gas compositions from respiratory exhalation. The said mass flow sensor shall be further preferably having a pair of thermal pile as one of the sensing elements that shall also be used as a trigger for the power switch when the inhalation is detected. The said mass flow sensor shall be package inside the device at a specially designed flow channel and shall be pre-calibrated for the desired accuracy for both the inhalation flowrate and the said inhalation and exhalation carbon dioxide composition.

In another preferred embodiment, the present disclosed device shall have a control electronics, that contains both the signal conditioning for the said silicon mass flow sensor integrated with the gas composition sensor and the feedback loop coupling the signals from silicon mass flow sensor and the heater power circuitry providing the power for the liquid vaporization. The said silicon mass flow sensor shall meter the air flowrate from the inhalation by the user. Due to the fast response time of the silicon mass flow sensor, the said thermal pile sensing element can be pre-set at a threshold that will be serving as a trigger for powering on the device such that the trace flowrate generated from other possible sources such as natural wind, vibration or natural movement when pocketed with a user. After the flowrate is over the threshold, the metered flowrate shall be converted into a signal that will be transmitted to the control circuitry for the heater of the vaporizer such that the power level can be adjusted according to the metered flowrate. In this configuration, the generated vapor amount shall be similar to those in a traditional smoking process where a larger inhalation shall result in a heavier smoke.

In another preferred embodiment, the disclosed device shall be able to measure the exhalation flowrate via the said silicon mass flow sensor. The exhalation flowrate can be further correlated to the peak flowrate of the respiratory profile. The measured data shall be informed to the user on a local display or via the wireless relay to a mobile device on which the data can be stored and analyzed. Such information shall remind the users who may have respiratory theses such as asthma or chronic obstructive pulmonary dieses to timely take necessary medication after the measured data indicated the abnormality. Further, this invented device shall be able to serve as a medication vapor generator that is needed by the medications such as for asthma, for example the budesonide inhaler. The dual function of the said device shall be particularly beneficial for the users with the said health conditions.

In another preferred embodiment, the said device shall have the capability of measurement of the spas concentration for the exhalation. The gas concentration shall include the carbon dioxide and oxygen concentration that shall be measured via the carbon dioxide and oxygen concentration sensors integrated with the said silicon mass flow sensor. The carbon dioxide concentration can be obtained with the measurement of the thermal conductivity and thermal capacitance of the respiratory exhalation while the oxygen concentration shall be obtained with an individual oxygen sensor that shall be also made via a MEMS process for a fast response time and small form factor as well as low power consumption such that the measurement can be achieved in the time frame of the exhalation. The sensing materials of the oxygen sensor is preferred to be the yttrium stabilized zirconia oxide as disclosed earlier by the inventors (US Patent Pub. No. 2014/0318960). The ratio of the oxygen to carbon dioxide or the respiratory exchange ratio shall be a direct indication of the user's metabolism current status that shall be preferably shown on a local display with the said device as the direct information for the user or the corresponding data shall be transmitted wirelessly to a mobile device for records and additional analysis. The data shall be further relayed from the mobile device to the designated cloud for big data process that shall be beneficial for public health information.

In yet another preferred embodiment, the data transmitted via the disclosed device shall be preferred to be relayed to the designated cloud for big data process upon the first approval by the user for privacy and data security purpose. The relayed information shall be preferred to be the daily operation time and frequency of the device, the geographic information, the exhalation respiratory flowrate as well as the respiratory exchange ratio. These data shall be further analyzed and formulated into the public health data domain, and would be preferred to feedback to the data provider such that the health status and alert could be applied to the adjustment for the lifestyle in the device consumption.

The present disclosure provides a close simulation to the experience of a traditional cigarette while adds the value for the user to monitor his/her own respiratory health status which shall further facilitate the lifestyle of the user. The said disclosure in particular after approval by the user shall relay the individual data into the designated cloud for big data process that shall be beneficial for the improvement of the public health conditions for the respiratory related diseases. These and other objectives of the present disclosure shall become readily apparent upon further review of the following drawings and specifications. And additionally for those with the knowledge of the art, the device could be further utilized for diagnosis of respiratory related diseases with additional precision sensors that could measure the trace chemicals which would be the markers of the known diseases.

### BRIEF DESCRIPTIONS OF THE DRAWINGS

FIG. 1 is the graphic presentation of the smart electronics vaporizer, showing the basic components of the device which includes three chambers, i.e. the vaporizer and inhalation/exhalation chamber, the sensing and electronic control chamber, and the power supply chamber with the display.

FIG. 2(a) is the detailed graphic presentation of the vaporizer and inhalation/exhalation part of the smart device that shows each of the components.

FIG. 2(b) is an alternative design for the access channel.

FIG. 3(a) is sensing chamber of the said smart device including the control electronics.

FIG. 3(b) is an alternative design of the said chamber.

FIG. 3(c) is the graphic presentation for the micro-machined silicon thermopile flow sensor.

FIG. 4(a) is the graphic presentation of power supply chamber.

FIG. 4(b) is the graphic presentation of three rectangular LED lights.

FIG. 4(c) is the graphic presentation of three concentric circular band LED lights.

FIG. 5 is the process flow of the control schematics of the smart device.

### DETAILED DESCRIPTION OF THE PREFERRED EMBODIMENTS

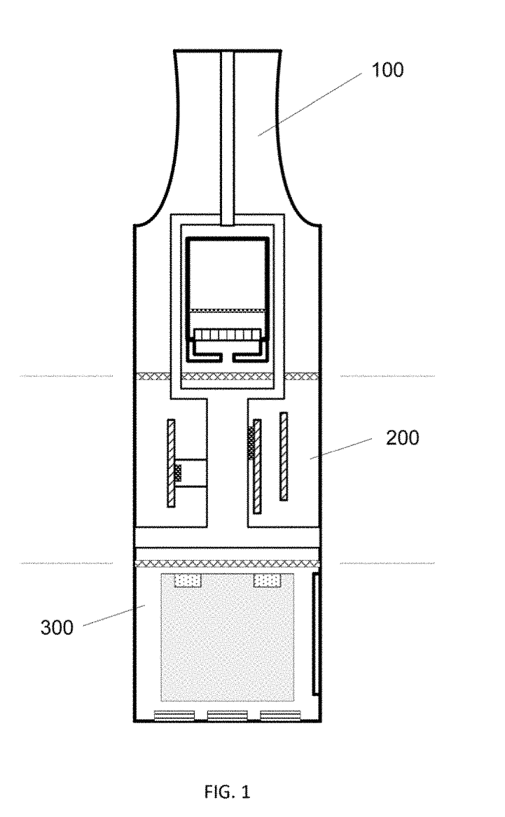

The preferred assembly of the said smart electronic vaporizer of the said invention is shown in FIG. 1. The said smart vaporizer shall be composed of three chambers. The vaporizer chamber (100) also contains the flow path for inhalation and exhalation, and is separated with sealing to the sensing chamber (200) where both inhalation and exhalation flow rate and gas composition are measured and analyzed with the on-board electronics and processed by the control electronics inside the same chamber. The power supply chamber (300) provide the rechargeable power to the said smart electronic vaporizer as well as the local display for the measured information together with a visual indicator of the said vaporizer status.

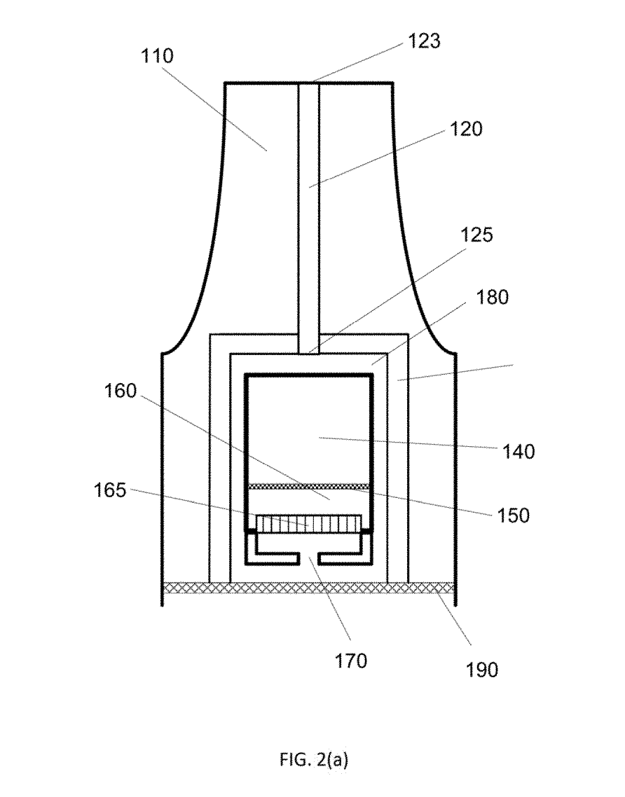

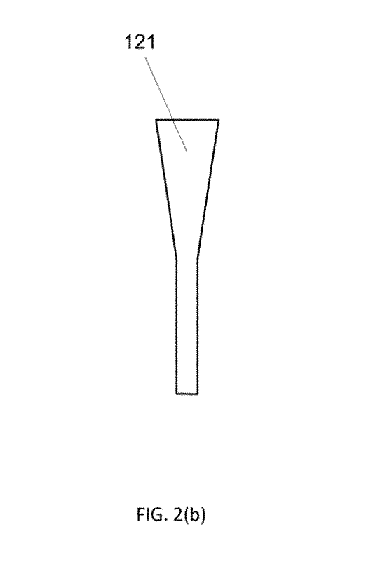

The electronic vaporizer chamber 100 is composed of several components for access path for inhalation and exhalation as well as those components for the smart vaporization process. The detailed description of the chamber can be seed from FIG. 2(a) and FIG. 2(b). The mouth piece 110 is preferred made of hygienic plastics or hygienic wood for the health safety of the normal requirements for such devices. The inhalation exhalation access channel 120 can be made by molding in case of hygienic plastics or machined for wood. It is preferred that the channel shall be made smooth at its channel walls, with either rectangular or circular shape, but preferably a circular shape. The access channel is further preferably having a shape (121) as shown in FIG. 2(b) where the opening at the part close to the outside access 123 shall be larger than that inside connected to the exhalation and air intake path 130 as well as the vapor inhalation entrance 125. Such a shape shall allow an efficient inhalation and exhalation supply while provide the gas flow stability. The liquid container 140 shall be made of preferably glass materials that shall be survived under high temperature. The single direction permeable mesh 150 separates the liquid from the vaporizing process while allows the liquid flow into the vaporizing process space 160 in which the heating elements 165 is placed. The heating elements 165 shall be made of hygienic materials such as noble metal platinum. Further there should not be any chemical reactions between the heating elements and the liquid to be vaporized. The permeable mesh 150 could also be an actuated valve that shall be controlled by the control electronics inside the sensing chamber 200. The said valve shall be opened to the desired position in connection to the controlled power of the heating elements, which would further be related to the inhalation strength measured by the air flow sensor inside the sensing chamber 200 such that the actuated valve 150 shall allow the desired amount of liquid into the vaporizing chamber 160 to supply the desired amount of vapor to the consumer through the dispenser nozzle 170 and the vapor supply channel 180. Finally seal materials 190 shall be used to separate the vaporizer chamber from other chambers such that any liquid would be contained inside the vaporizer chamber only.

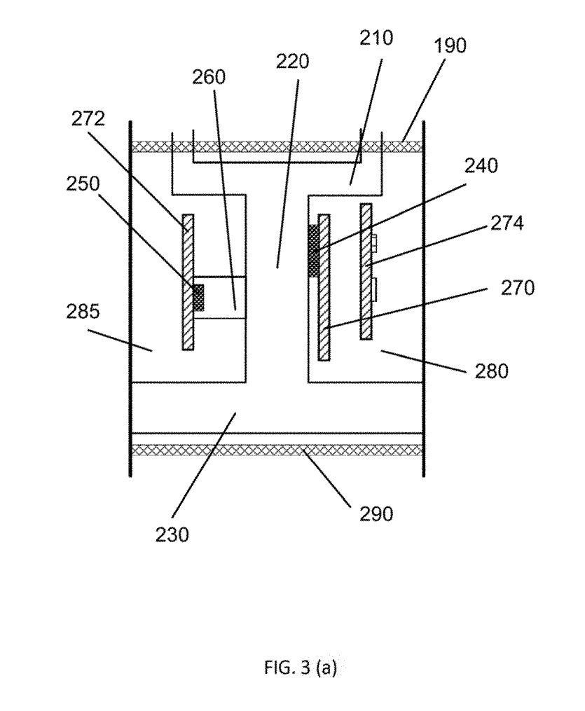

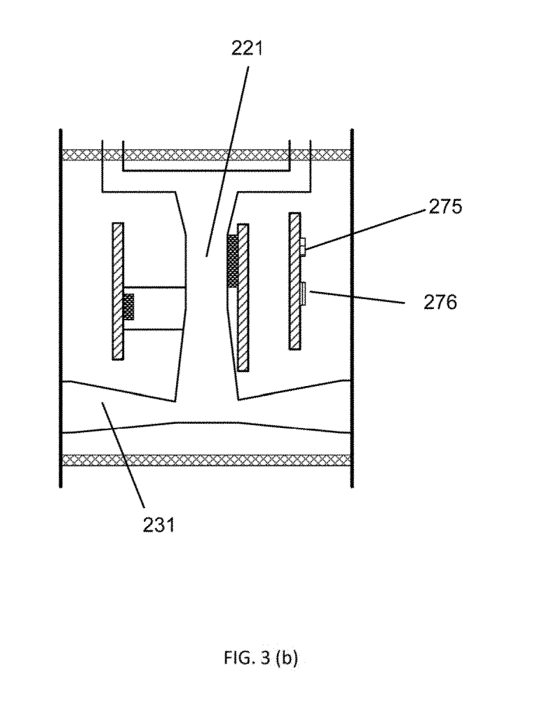

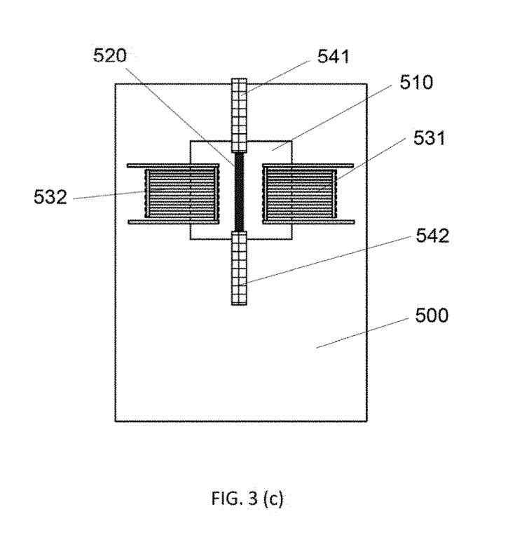

FIG. 3 presents the configuration and structure of the sensing chamber 200. The sensing chamber 200 is connected to the vaporizer chamber via the air flow channel 210. The channel 220 is where the sensing elements are placed and it is further connected to air intake and exhalation channel 230 where the outside air could flow in while the exhalation respiratory gases could be expelled out. The silicon mass flow sensor 240 is preferably installed at the wall of the flow channel 220 while the gas composition sensor 250 requires a gas analysis chamber 260 which is preferably an cavity made through an opening in the flow channel 220 such that the flow induced interference shall be minimized. The silicon mass flow sensor 240 shall preferably have the wake up trigger made of thermal piles that shall not require an external power to operate. For efficiency in data process or fast response time and requirements for small form factor as well as requirements for low power consumptions, the silicon mass flow sensor 240 is further preferably integrated with the carbon dioxide gas sensing elements which could be made of the thermal conductivity and thermal capacitance sensors. The electronics signal of the silicon mass flow sensor is processed by the signal condition printed-circuit-board (PCB) 270 and further connected the control electronics PCB 274. The gas composition sensor 250 shall be preferably made of a MEMS process on silicon substrates as well for the same reasons as those for the silicon mass flow sensor. The gas composition sensor shall further preferably be able to measure the oxygen concentration in exhalation process. The preferred sensing elements include yttrium stabilized zirconia oxide or the metal doped (such as molybdenum) tin oxide. The electronic signal from gas composition sensor is further processed by the gas composition sensor signal conditioning PCB 272 which then relax s the signal to control electronics PCB 274 on which there are a central processing unit (276) to process the signals and a communication interface wireless module (275) such as a to power Bluetooth module. The control electronics PCB shall preferably have a wireless transmission module that could further relay the information to a mobile device for additional data process and records. All these electronics are placed inside enclosures 280 and 285 such that the electrical isolation and safety can be assured. The seal materials 290 shall be used to separate the sensing chamber from power supply chambers such that change or re-charge the battery shall not have any impact to the sensing chamber configurations.

The details micro-machined or MEMS silicon mass flow sensor is ideally a thermal pile flow sensor as exhibited in FIG. 3(c). The sensor can be made on a silicon substrate (500) inhere a suspended membrane (510) made of silicon nitride supports the micro-heater (520) and the hot-cold junction piles or thermal piles (531 and 532). The electrical connections (541 and 542) are made of gold or aluminum. During the operation, the fluid media passing throng the sensor will bring the changes to the temperature field created by the micro-heater and the thermal piles will register such changes and correlate to the flow rate of the fluid media.

Alternatively, the flow channel 220 shall be preferably made into a shape (221) as indicated by FIG. 3(b). This preferred embodiment shall be helpful for improvement of flow stability during the measurements for both the inhalation and exhalation flowrate as well as measurement of gas composition, in particular for the detection of the transitional status from the sleeping mode to operation where only very low flow rate could be measured. Similarly, the air intake and exhalation channel 230 is preferably made into the shape of 231 for the flow stability configuration.

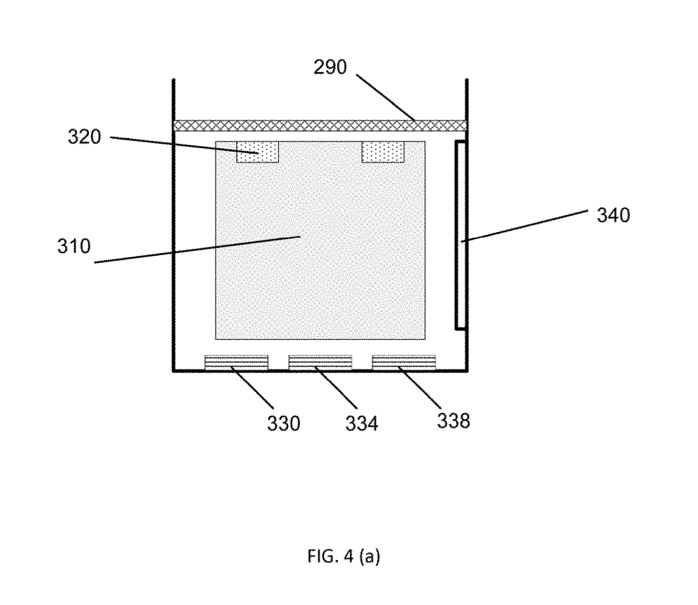

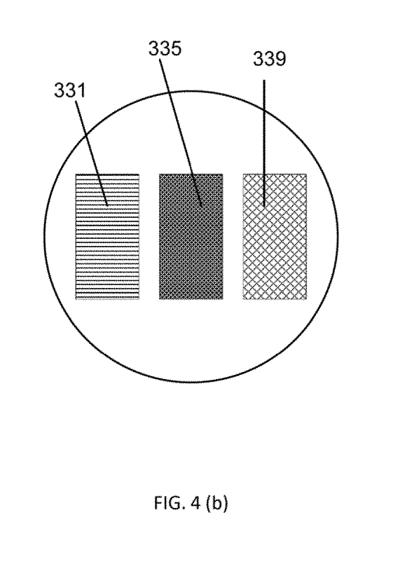

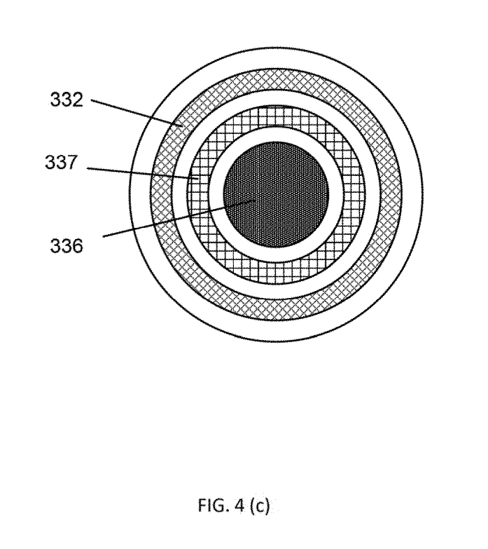

The power supply chamber show in FIG. 4(a) shall contain the battery 310 with electrodes 320 for supplying the power to the control electronics and signal conditioning circuitries as well as the heating elements for the vaporizer. The battery is preferably a rechargeable lithium ion battery but it could be an alternative device containing energy harvesters which could collect the energy from natural sources such as vibration and convert such into electrical power. A local display 340 shall alert the necessary measured digital information to the device user. The display is preferably made of colored active-matrix organic light-emitting diode (AMOLED), but it could be the simple liquid crystal display (LCD). The additional three light alert 330/334/338 shall be preferably made of colored light-emitting diode (LED) lights that shall indicate the operation status of the said vaporizer device. Each of the LED could have different color or same color with different light strength and in the shape of a square or circular. These three LED lights could be alternatively in the form of rectangular 331/335/339 (FIG. 4(b)) or in the form of concentric circular band 332/336/337 (FIG. 4(c)).

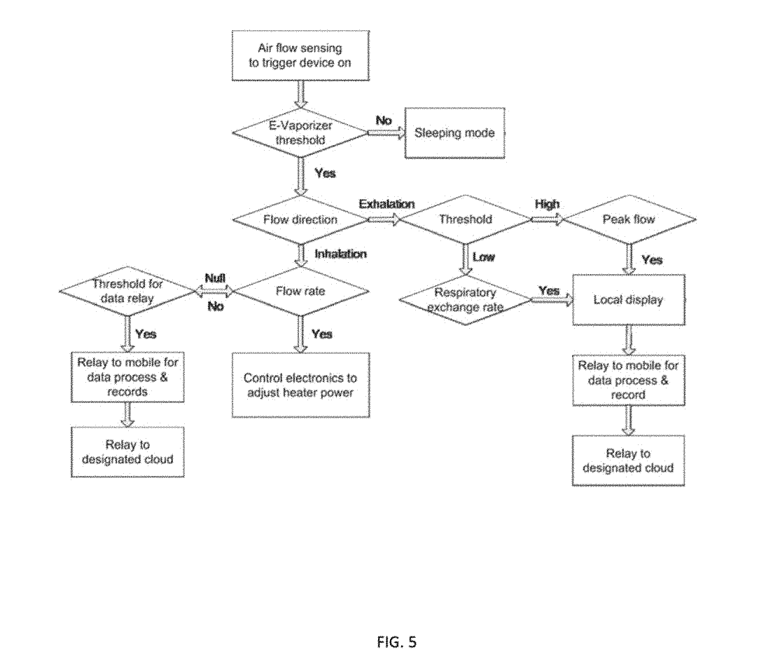

For the preferred embodiments, the actual sensing and control scheme of the said smart vaporizer device is shown in FIG. 5. At the normal status, the said device shall be in the sleeping mode for maximum power saving. When the user starts to operate the device, the user shall start to inhale from the mouth piece 110, the air intake from the air intake channel 230 shall wake up the silicon air mass flow sensor via the detection of the temperature differences due to the air flow by the wake up sensors on the same silicon mass flow sensor. Such a wake up sensor could be made of a thermal couple that shall not require any external power to operate which shall be preferably for the battery power saving operation. After the sensor wakes up air mass flow sensor, the air mass flow sensor shall meter the flowrate of the inhalation and compare it to the pre-set value for the threshold of the vaporizer operation. This threshold shall allow the device from malfunction due to vibration or other movement induced trace air circulation inside the said device. In case that the measured flowrate is lower than that of the threshold set for the vaporizer to operate, the said device shall be kept in the sleeping mode.

For the preferred embodiment, when the flowrate shall be higher than the pre-set value of the threshold for the vaporizer, the silicon mass flow sensor shall further measure the direction of the flow. In case that the flow direction is measured to be inhalation, then the flowrate values shall be sent to the control electronics 274 that shall manage the vaporizer heating circuitry including the permeable mesh or the actuated valve 150 to the liquid such that amount of liquid and the power supplied for heating elements 165 for vaporization process would be desired. For the ultimate performance, the parameters of the threshold and the flowrate related vaporized process could be pre-calibrated and stored in the control electronics. The silicon mass flow sensor shall then continuously meter the flowrate of the inhalation and provide the value to the control electronics, In case that a null value or value below the threshold that allow to trigger the vaporizer shall be detected, the value shall be sent to the control electronics and the value shall be compared to the pre-set threshold (duration) for the conclusion of the device usage. Once it is confirmed that the device ceased to operation, the control electronics shall trigger a data relay process to the pre-paired mobile device via the wireless module embedded in the control electronics. The preferred data to relay is preferably but not limited to the duration of the device usage, the maximum and/or integrated flowrate or consumption of the vapor as well as the calendar information. The paired mobile device shall then further relay the information at the pre-set time period to the designated cloud for big data process and sharing.

For the preferred embodiment, in case that the flowrate measured by the silicon mass flow sensor shall be higher that the vaporizer threshold, and an exhalation direction shall be measured, then the value shall be sent to the control electronics for comparison with the flowrate threshold that shall be set for respiratory disease or respiratory metabolism measurement, in case that the threshold shall indicate a respiratory disease such as asthma measurement, the measured value shall be compared to the re-stored value in the electronics for individual's peak flow and further relay the data to the local display. In the case for asthma detection, if the peak flow measured shall be lower than those for warning level, the corresponding warning sign shall also be shown on the local display. The control electronics then shall automatically relay the information to the designated mobile device for further data process and record, and the paired mobile device shall then further relay the information at the pre-set time period to the designated cloud for big data process and sharing.

For the preferred embodiment, in case that the flowrate measured by the silicon mass flow sensor shall be higher that the vaporizer threshold, and an exhalation direction shall be measured, then the value shall be sent to the control electronics for comparison with the flowrate threshold that shall be set for respiratory disease or respiratory metabolism measurement. In case that the threshold shall indicate a respiratory metabolism measurement, the data shall be collected from the gas composition sensors for oxygen and carbon dioxide composition values. The ratio of these two gases or the respiratory exchange ratio shall be compared to the re-stored value in the electronics for individual normal ones and further relay the data to the local display. If the value measured shall be higher than those fur warning level, the corresponding warning sign shall also be shown on the local display. The control electronics then shall automatically relay the information to the designated mobile device for further data process and record, and the paired mobile device shall then further relay the information at the pre-set time period to the designated cloud for bid data process and sharing.

For the additional preferred embodiment, the said device for those in the art shall become readily and apparently could be further incorporated with addition gas composition analyzer and the data shall be available for use of detecting certain markers from individual's respiratory gaseous materials. It shall also be readily and apparently that the liquid for vaporizing shall not be limited to nicotine but could also those containing healthy elements. The said smart electronic vaporizer shall then be a mean for lifestyle.

1. A smart electronic vaporizer device with a capability to simulate a traditional cigarette experience and provide individual's respiratory health status data comprising:
A micro-machined silicon mass flow sensor which is connected to a flow sensor control electronics circuit and has a capability for metering gas mass flow rate as well as thermal values of inhalation breath from users;
A micro-machined silicon thermopile flow sensor with sensing elements made with thermopiles wherein the micro-machined silicon thermopile flow sensor is used to sense a very low flow rate of inhaling breath from users and provide a trigger signal for power supply to turn on the smart electronic vaporizer device from standby;
A micro-machined silicon gas composition sensor which is attached to a gas composition control electronic circuit and has a capability to analyze gas concentration of oxygen and carbon dioxide;
A liquid container that is used to store combustion liquid; and, wherein a liquid vapor could be used to imitate a smoking feeling like traditional cigarettes;
A vaporizing heating element wherein power level of the vaporizing heating element is controlled by a heating element control electronic according to inhalation flow rate or strength measured by the silicon mass flow sensor;
A flow passage for inhalation and exhalation wherein the micro-machined silicon mass flow sensor is deployed in sidewall of the flow passage; and
A flow sensor control electronics circuit with a wireless communication module which can be used to connect to a device with wireless communication capability for data transmission;
wherein a mechanical enclosure is used to housing components for accomplishing vaporizing process and respiratory data measurement.

2. The smart electronic vaporizer device of claim 1 wherein the micro-machined silicon mass flow sensor is able to measure gas flow rate with pressure range up to 10 bar and a flow speed range of 0.003·90 m/sec.

3. The smart electronic vaporizer device of claim 1 wherein the micro-machined silicon thermopile flow sensor has sensing elements made of thermopiles that will not require external power to operate and could detect very low flow for lower than 0.03 m/sec wherein the thermopiles could be deployed to trigger a signal for flow measurement.

4. The smart electronic vaporizer device of claim 1 wherein the micro-machined silicon gas composition sensors incorporates a sensing element made of yttrium stabilized zirconia oxide for oxygen composition measurement that is able to sense oxygen concentration from 0˜50% vol.; wherein the gas composition sensor further have another sensing element made of tin oxide doped with molybdenum, which is able to provide concentration of carbon dioxide from 0˜10% vol.

5. The smart electronic vaporizer device of claim 1 wherein the micro-machined silicon mass flow sensor is installed at sidewall of the flow passage while the micro-machined silicon gas composition sensor is installed inside a cavity connected to the flow package of the smart electronic vaporizer device,

6. The smart electronic vaporizer device of claim 1 wherein the liquid container is made of hygienic materials chosen from medical grade glass, or high temperature medical plastics; wherein liquids in the liquid container is water-based solutions with nicotine ingredient.

7. The smart electronic vaporizer device of claim 1 wherein the vaporizing heating element is made of hygienic materials chosen from platinum or heavy doped polysilicon; wherein the vaporizing heating element is powered by the electronics control circuit which the heating power is adjusted in proportion to flow rate of inhalation breath measured by the micro-machined silicon mass flow sensor.

8. The smart electronic vaporizer device of claim 1 wherein the heating element control electronic is coupled to control liquid supply from the liquid container and whole capability of vaporizing process by controlling a permeable mesh or an actuated-valve opening.

9. The, smart electronic vaporizer device of claim 1 wherein the flow passage is made of hygienic materials and in a shape of venturi profile in order to maintain maximum flow stability inside the flow; wherein the flow passage is as well able to provide a peak flow measurement capability with a flow resistance. 10, The smart electronic vaporizer device of claim 1 wherein the communication module is able to be paired by designated mobile devices for data security, data processing and recordings.

11. The smart electronic vaporizer device of claim 1 wherein the communication module is equipped with a capability to further relay the data from the mobile devices to a designated cloud data center for big data processing and sharing,

12. The smart electronic vaporizer device of claim 1 wherein the flow sensor control electronics circuit has a protocol to further transmit data from the smart phone to a designated cloud service for big data process and sharing.

13. The smart electronic vaporizer device of claim 1 wherein the flow sensor control electronics circuit has a central process unit that is capable of measuring respiratory health data from the micro-machined silicon gas composition sensors; wherein the central process unit is able to transmit data to local display with necessary warnings to users.
